(19) United States
(12) Patent Application Publication
(10) Pub. No.: US 2023/0346453 A1
(43) Pub. Date: Nov. 2, 2023

(54) SURGICAL GENERATOR HAVING REMOTE-CONTROLLED FUNCTIONALITY

(71) Applicant: OLYMPUS WINTER & IBE GMBH, Hamburg (DE)

(72) Inventors: Jens KRÜGER, Zeuthen (DE); Anne KWIK, Berlin (DE)

(73) Assignee: OLYMPUS WINTER & IBE GMBH, Hamburg (DE)

(21) Appl. No.: 18/129,425

(22) Filed: Mar. 31, 2023

Related U.S. Application Data
(60) Provisional application No. 63/326,059, filed on Mar. 31, 2022.

(51) Int. Cl. A61B 18/12; G08C 23/00

(52) U.S. Cl. CPC A61B 18/1206; G08C 23/00; A61B 2018/00178

(57) ABSTRACT

A surgical generator providing a high-frequency alternating voltage to a surgical instrument. It includes a control unit and a user interface connected thereto. The control unit is configured for controlling the surgical generator based on a set of functions. A signaling interface is provided for optical signaling in a bidirectional manner to and from the remote control. The signaling interface configures the control unit and/or the surgical generator dependent on communication from the remote control. Thereby the surgical generator can be re-configured by limiting certain functions for different fields of application by the remote control. Optical signaling avoids risks of radio transmission. It is an efficient short range communication usable within same room only, providing safety against external access. Thus, re-configuration can be accomplished in a safe and cost-effective manner.

The invention relates to a surgical generator having a functionality defined by a set of functions. Specifically, the surgical generator is configured to output a high-frequency alternating voltage to a surgical instrument comprising a control unit and an inverter generating high-frequency alternating voltage which is output to an output socket for connection of said surgical instrument, and a user interface for user input and output being functionally connected to the control unit, the control unit being configured for controlling the surgical generator based on a set of functions.

Present surgical generators typically feature a rather complex functionality comprising a large set of functions. Thereby, the surgical generators are enabled to cope with various applications of said surgical generator, e.g. in the field of NET (nose, ears, throat), gastroenterology, urology, etc. most of which requiring special functions. In practical use, a surgical generator is usually employed for one of these application fields requiring only a subset of the total set of functions (functionality) the surgical generator is capable of. For a specific surgical generator, there are typically some functions which are essentially superfluous with respect to the application field said surgical generator is actually used for. Presenting all these functions including the superfluous functions to the surgeon make selection of useful functions more cumbersome. Further, there is a risk of cluttering the user interface which is detrimental for ease and safety of use.

It was envisaged to employ remote control units allowing the user in the operating theater to configure a surgical generator according to the relevant application field, namely by limiting certain functionalities which are rarely of no use in said application field. For ease of operation of such remote control units, a wireless interface is preferable. However, providing radio links for operating safety critical devices, like a surgical generator, in an actual operation theater is to be avoided due to regulatory constraints and safety considerations. Further, using radio links, there is a risk that unwarranted control could be made from outside of the operation room which could pose a serious risk for safety and health of the patient.

Accordingly, there is a need for an improved way to ease remote control in the operating theatre while reducing the risk of unwanted tampering from outside.

The solution according to the invention resides in the features of the independent claims. Advantageous embodiments are the subject matter of the dependent claims.

In a surgical generator configured to output a high-frequency alternating voltage to a surgical instrument comprising a control unit and an inverter generating high-frequency alternating voltage which is output to an output socket for connection of said surgical instrument, and a user interface for user input and output being functionally connected to the control unit, the control unit being configured for controlling the surgical generator based on a set of functions, it is provided according to the invention a signaling interface for communication of the control unit with a remote control, said signaling interface being configured for optical signaling in a bidirectional manner to and from the remote control, said signaling interface being connected to the control unit and being adapted to configure the control unit and/or the surgical generator dependent on communication received from the remote control.

Configuring of the surgical generator is understood to comprise defining which functions, set of functions, functionality and/or mode of operation are available to and selectable by the user.

According to the invention, a configuration of the surgical generator and its control unit can be adapted or altered according to the intended application field of said surgical generator. For example, if a surgical generator is to be used for the application field of gastroenterology then certain functions and/or model not belonging to this field, like e.g. special functions to be used for urology or gynecology, will be locked and therefore the configuration of the surgical generator will adapted to said another or new application field. Specifically, said adaption could be made by restricting certain functions of the set of functions the surgical generator is capable of. Similarly, if a surgical generator is to be used for the application field of ENT (ear, nose, throat) and other special functions not belonging to this field will be limited and accordingly locked in their control unit. Configuring the surgical generator by limiting the available set of functions to the specific application field is a great benefit to the users, as thereby complexity of the surgical generator and in particular its user interface can be reduced. Thereby, any risk of inadvertent selection of a function or mode of operation that is not appropriate is avoided. Further, cluttering of the user interface is reduced, thereby enhancing ease and reliability of operation. Risk of inadvertent errors are significantly reduced, thereby enhancing safety for the patient.

The invention provides for a bidirectional communication interface with a remote control, for example a smart phone acting as a short-range remote control. By using optical signaling, the disadvantages and the regulatory complexities of radio communications are effectively avoided. Further, such optical communication means is effectively a short range communication means operable within the same room only, since walls and doors block the optical signal. Thereby, only a person present in the same room is effectively enabled to perform the remote control, i.e. a person that has physical access to the surgical generator anyway. Therefore, no additional security risk is introduced by the invention.

A further advantage of the invention is that little or no additional hardware is required. Most surgical generators already have means capable of signaling optical information like displays that could also be employed as a transmitting channel for the present invention. Conversely, many surgical generator also have a light sensor in order to determine ambient light conditions which may also double as a sensor for a receiving channel of the present invention. Thereby, the present invention can be added at surgical generators with little or no additional hardware cost.

Further, the invention has the benefit of not requiring specific dedicated remote control devices. Any smart phone is enabled to receive optical signaling data by virtue of its camera. Conversely, by virtue of its display lighting, its flash or its message signaling LED (if present), the smart phone is enabled to transmit optical signaling data back to the surgical generator. Therefore, any smart phone can be employed as a low-cost remote control in conjunction with an appropriate app (application program). This has the additional benefit of any authentication required by said app adds an additional layer of protection against unauthorized configuration action.

The invention thus provides an economical, secure and radio frequency interference (RFI)-free bidirectional communication interface between the surgical generator and the remote control, in particular a ubiquitous staple smart phone to be used as a low-cost remote control.

Preferably, the optical signaling interface comprises two distinct channels, a first, transmitting channel for outbound communication to and a second, receiving channel for inbound communication from the remote control. By using such distinct channels, as opposed to a single bidirectional channel, different means could be used for transmitting and receiving. This facilitates using already present components of the surgical generator to be employed for the optical signaling interface, thereby making double use of these components and minimizing additional efforts.

Advantageously, to this end the first, transmitting channel comprises a data field on a display of the surgical generator, said data field showing configuration data of the surgical generator in a machine readable coded format, preferably a QR-code, barcode or movable graphic code. By using such a data field, preferably on an already existing display of the surgical generator, information can be sent out in an efficient manner to be read visually by the remote control operated by the user of the surgical generator. Presenting that configuration data in the data field features a further advantage that it is now visible to the user that configuration data is shown and the corresponding process is active, thereby prompting the user that appropriate action is necessary. Further, since surgical generators typically are already provided with a capable display, no additional hardware is required. Said data field can optionally also display movable graphic code, e.g. so called flicker code (known in the field of online banking for controlling TAN generators). These allow a rather high speed of data transmission and/or larger amount of data in the same time, thereby enabling usage of codes having a higher redundancy for further improved safety and security.

Further advantageously, to this end the second, receiving channel comprises a photosensitive sensor arranged at a housing of the surgical generator. The photosensitive sensor may be a sensor configured to determine external lighting conditions, wherein “external” means lighting conditions in the surroundings of the surgical generator, e.g. room brightness. However, the sensor could also be a sensor dedicated to receive optical data, like a sensor for infrared light. In the latter case, sensitivity could be higher which is advantageous in case of a rather weak optical signal is to be used, like a light emitted of the message LED of the remote control; additionally this decouples the receiving from room lighting conditions. Alternatively, a camera may be used as photosensitive sensor also.

Preferably, the photosensitive sensor is connected to a signal filter configured to extract a modulated signal from an output of the photosensitive sensor. Using a modulated signal allows decoupling from actual room lighting conditions and changes of room lighting conditions. Thereby, it allows to employ the same sensor for ambient lighting detection as well as for receiving the optical signaling by virtue of the modulated signal. This double use avoids the expenditure of providing an additional sensor. Further, using a signal filter for extracting the modulated signal increases robustness of optical data transmission against random disturbances. Further preferably, the signal filter comprises a flicker filter. Thereby, signal disturbances induced by flickering ambient lighting, as it is of experienced under artificial lighting conditions, can be reduced. The flicker filter reduces disturbances caused by the frequency of the electrical grid (50 Hz / 60 Hz and harmonics thereof); this line frequency induced flicker must not be confused with flicker code that is intentionally shown on a display in order to convey coded data.

In an advantageous embodiment, an output of the signal filter is supplied to a demodulator device of the signaling interface. Thereby, the modulated signal is effectively extracted and can be presented as a directly usable baseband data signal to the control unit.

The invention further relates to a system comprising the surgical generator as described above and a remote control, preferably a smart phone. The invention yet further relates to a method according to the features of independent method claim. In order to avoid unnecessary repetition, for further explanation, reference is made to the above description which applies to the system and method likewise.

The invention is explained in more detail below with reference to an advantageous exemplary embodiment. In the figures:

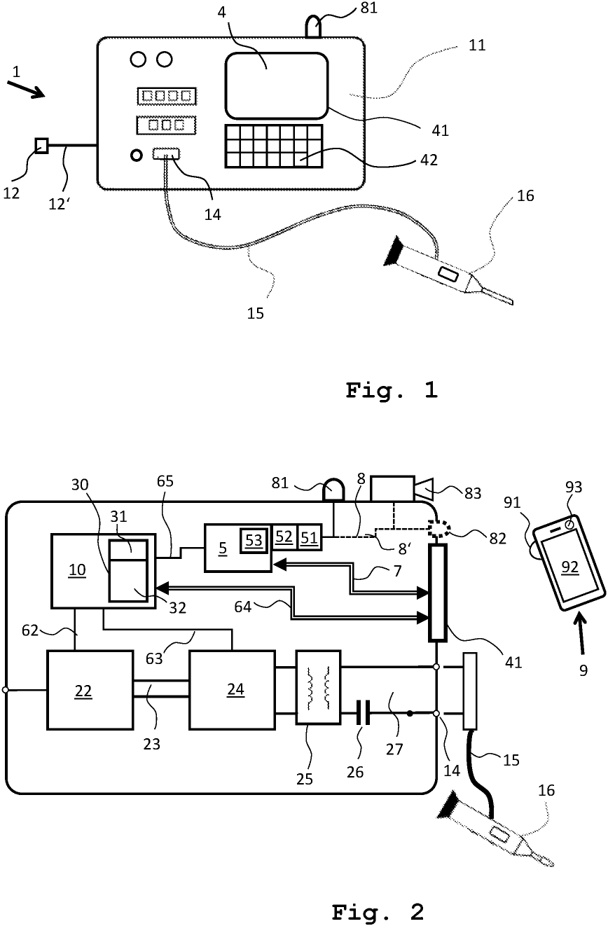

FIG. 1 shows a surgical generator according to an exemplary embodiment with an attached electrosurgical instrument;

FIG. 2 shows a block diagram of the surgical generator shown in FIG. 1 and a remote control;

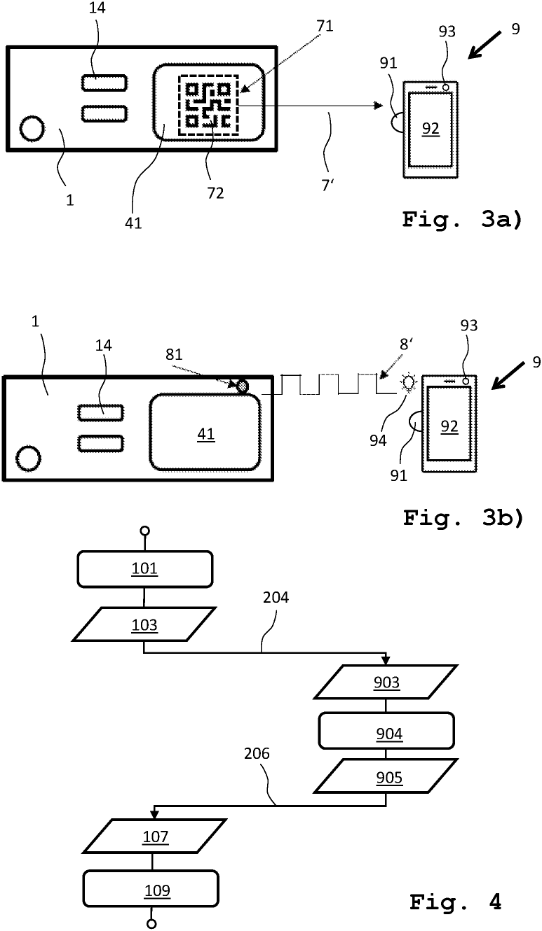

FIGS. 3a, b show a transmitting channel and a receiving channel, respectively, of an optical signaling interface to a remote control; and

FIG. 4 shows a flow diagram of a method according to the invention.

In the illustrated embodiment, the surgical generator is an electrosurgical generator identified as a whole by reference numeral 1. The electrosurgical generator comprises a housing 11 provided with an output socket 14 for an electrosurgical instrument 16 which, in the exemplary embodiment illustrated, is an electrocautery. It is connected via a high-voltage connecting cable 15 to the output socket 14 of the electrosurgical generator 1. A mains connecting cable 12′ connected to a plug 12, which can be connected to a public electricity grid or other suitable means of electrical supply, is provided for the supply of electrical power to the electrosurgical generator 1.

A functional block diagram of the electrosurgical generator 1 is illustrated in FIG. 2. It comprises in the housing 11a power supply unit 22 that is supplied with electrical power by the mains connecting cable 12′ (see FIG. 1). The power supply unit 22 is a high-voltage power supply unit (HVPS). It comprises a rectifier and feeds a DC link 23 with direct voltage, the DC link 23 supplying an inverter 24. The inverter 24 generates high-frequency alternating current in the high-voltage range of a few kilovolts. The high-frequency high voltage output is fed via an isolation transformer 25 to the output socket 14 via an output line 27 comprising a blocking capacitor 26. This is generally known in the art and will not be further explained for the sake of brevity.

Operation of the electrosurgical generator 1 is controlled by a control unit 10 which is connected to the power supply unit 22 and the inverter 24 by means of signaling lines 62, 63, respectively. The control unit 10 operates the electrosurgical generator 1 based on a set of functions 32 which are stored in a function memory 30. The functions 32 define operating characteristics and modes of the electrosurgical generator 1. According to an application field for the electrosurgical generator 1 at hand, certain functions may be restricted from actual use, these functions are called limited functions 31.

A user interface 4 for user input and output is connected to the control unit 10 by means of a data bus 64. The user interface 4 comprises a display 41 presenting information on the operating status or configuration of the electrosurgical generator 1 to the user and a keyboard 42 which may also be a touchscreen input device in order to enable the user to enter data and commands for the control unit 10. On the housing 11 is mounted an ambient light photosensitive sensor 81. It is configured to detect brightness of ambient light. Its output sensor signal is fed to the control unit 10 in order to determine a brightness of the display 41 of the user interface 4 dependent on ambient light conditions.

Signaling interface 5 is connected to the control unit 10 via signaling line 65. Further, the signaling interface 5 comprises two channels, a first transmitting channel 7 by virtue of which it is connected with the display 41, and a second receiving channel 8 connected to the photosensitive sensor 81 configured as an ambient light sensor. Specifically, the photosensitive sensor 81 is connected to a filter 51 which is configured to extract a modulated signal from an output signal of the photosensitive sensor 81. Thereby, a modulated signal can be extracted from the output signal of the photosensitive sensor 81, while the photosensitive sensor 81 still fulfills its main purpose of providing information about ambient light conditions to the control unit 10.

Selection of functions 31 which shall be limited from actual is not an everyday task. It is performed when setting up the electrosurgical generator 1 for its application field in which it is to be actually used, for example in a hospital. Those functions 31 that are limited shall not be presented to the user on the display 41 in order to avoid cluttering of said display and to avoid any risk of inadvertently selecting such a function. However, a restriction of the functions 31 to be limited from actual use is only to be made by an authorized user in an authorized manner, and that authorized user shall be physically present at the electrosurgical generator 1. Said user is equipped with a remote control which is typically a staple smart phone 9 running a specific app that is provided by the manufacturer of the electrosurgical generator 1. The smart phone 9 comprises a display 92, a camera 91, a flash 94 and optionally a signaling LED 93 configured to signal incoming messages to a user.

In order to configure the electrosurgical generator 1 to its application field and to select functions 32 to be restricted, the transmitting channel 7 transmits data to a data field 71 on the display 41. Thereby, said data field 71 shows visually machine-readable codes representing configuration data of the surgical generator 1 in a machine readable coded form, preferably as a QR-code 72. Thereby, a transmission channel 7 is formed, the flow of transmitted data being represented by arrow 7′ in FIG. 3a). Using the camera 91 of the smart phone 9, that visual readable machine code in the form of a QR-code 72 is read from the data field 71 and its data is conveyed to a special app running on the smart phone 9. Thereby, the smart phone 9 is enabled to accept user selections on functions 32 that are to be limited. Once the selection is made, the smart phone 9 activates a light source like a message LED 93, a background light of the display 92 or an inbuilt flash 94 such that it will emit light in a modulated manner to be received by the electrosurgical generator 1. This completes the receiving channel 8, the flow of modulated data being received is represented by square-waved line 8′ symbolizing digital data signals. Reception is to be made by the photosensitive ambient light sensor 81, and the received data will be extracted from its output signal by means of the signal filter 51 and its optional flicker filter 52 to remove signal disturbances induced by flickering ambient lighting, as it is of experienced under artificial lighting conditions. Then, the filtered signal is demodulated by a demodulator 53 and finally supplied to the signaling interface 5. This interface then transmits the data to the control unit 10, thereby effecting a configuration concerning the limited functions 32 as selected by the user on his smartphone 9.

Rather than the ambient light sensor 81 as a photosensitive sensor, also a special light sensor 82 may be used that is configured as a sensor for receiving optical signals. Further, it is also possible to employ a camera 83 to receive said optical signal, thereby enabling also an optional direct reading from the display 92 of the smart phone 9. Such a camera 83 is particularly useful for reading of more complex machine-readable codes shown on the display of the smartphone 9. Examples for such more complex machine-readable code are moving graphic codes like so called “flicker codes” (known in the banking field for controlling TAN generators), allowing a rather high speed of data transmission and/or usage of codes having higher redundancy for safety and security.

As all these are optical means of transmission and reception, issues with radio and radio interference are completely avoided. Further, since such optical signals do not travel through walls or doors, there is implicit safety by requiring physical presence of the person authorized to perform the configuration in the same room. Therefore, physical presence is required thereby achieving a similar or better degree of safety compared to a conventional configuration interface being nested in a menu structure of the electrosurgical generator.

The steps of the method to be performed in order to configure the electrosurgical generator 1 accordingly are shown in FIG. 4. In the first step 101, the configuration process is initiated on the electrosurgical generator 1. Then, the control unit 10 via the optical signaling interface 5 displays a data field 71 comprising visually machine-readable code, like said QR-code 72, in step 103. This establishes transmitting 204 of the optical signal via the transmission channel 7 to the smart phone 9. In a next step 903, said visual machine-readable code is read by the camera 91 of the smart phone 9. Processing and selection of appropriate function is performed on the smart phone in step 904. When completed, configuration data is then emitted from the smart phone 9 in step 905, e.g. by flashing a background light of the display 92, a messaging LED 93 or the flash 94 of the smart phone 9. The modulated data sent thereby via the receiving channel 8 is received in step 206 and read by the photosensitive sensor 81 in step 107 with subsequent demodulation. This data is then processed by the optical signaling interface 5 and outputted in step 109 to the control unit 10 in order to limit certain functions 31, thereby configuring the surgical generator 1.

1. A surgical generator configured to output a high-frequency alternating voltage to a surgical instrument comprising a control unit and an inverter generating high-frequency alternating voltage which is output to an output socket for connection of the surgical instrument, and a user interface for user input and output being functionally connected to the control unit, the control unit being configured for controlling the surgical generator based on a set of functions,
comprising
a signaling interface is provided for communication of the control unit with a remote control,the signaling interface being configured for optical signaling in a bidirectional manner to and from the remote control, the signaling interface being connected to the control unit and being adapted to configure the control unit and/or the surgical generator dependent on communication received from the remote control.

2. The surgical generator of claim 1, wherein the optical signaling interface comprises two distinct channels, a first, transmitting channel for outbound communication to and a second, receiving channel for inbound communication from the remote control.

3. The surgical generator of claim 2, wherein the first, transmitting channel comprises a data field on a display of the surgical generator, the data field showing configuration data of the surgical generator fB-in a machine readable coded format.

4. The surgical generator of claim 3, wherein the second, receiving channel comprises a photosensitive sensor arranged at a housing of the surgical generator to detect external lighting conditions.

5. The surgical generator of 4, wherein the photosensitive sensor is an ambient light sensor.

6. The surgical generator of claim 4, wherein the photosensitive sensor is an infrared sensor.

7. The surgical generator of claim 4, wherein the photosensitive sensor is a camera.

8. The surgical generator of claim 4, wherein the photosensitive sensor is connected to a signal filter configured to extract a modulated signal from an output of the photosensitive sensor.

9. The surgical generator of claim 8, wherein the signal filter comprises a flicker filter.

10. The surgical generator of 8, wherein an output of the signal filter is supplied to a demodulator device of the signaling interface.

11. The surgical generator of claim 1, wherein the signaling interface is adapted to restrict the set of functions available by the control unit.

12. A system comprising the surgical generator according to claim 1 and the remote control.

13. A method of configuring the surgical generator comprising a control unit and an inverter generating high-frequency alternating voltage which is output to a surgical instrument and a user interface for user input and output being functionally connected to the control unit, the control unit being configured for controlling the surgical generator based on a set of functions,
comprising
optical signaling of communication data to and from an remote control via an optical signaling interface,
configuring the control unit and/or the surgical generator dependent on communication data received from the remote control.

14. The method according to claim 13, comprising transmitting (-204-)-of communication data by data field (744 comprising configuration data of the surgical generator in a machine readable coded format.

15. The method according to claim 13, comprising receiving of communication data by means of a photosensitive sensor.

16. The method according to claim 13, wherein the remote control is employed for
reading data in the machine-readable coded format from the data field and
sending configuration data by means of a light-emitting device of the remote control, a message LED of the smart phone and/or by modulating brightness of a display of the smart phene-(9hphone.
